(19) United States
(12) Patent Application Publication
(10) Pub. No.: US 2024/0219131 A1
(43) Pub. Date: Jul. 4, 2024

(54) SYSTEM AND METHODS FOR DETECTING FAILURE OF HEAT EXCHANGERS

(71) Applicant: Pentair Water Pool and Spa, Inc., Cary, NC (US)

(72) Inventors: Keith Lewis, Moorpark, CA (US); Mitchell Bellamy, Sanford, NC (US); Charles Bulger, Falcon Heights, MN (US); Azur Dzindo, Raleigh, NC (US)

(21) Appl. No.: 18/397,346

(22) Filed: Dec. 27, 2023

Related U.S. Application Data
(60) Provisional application No. 63/477,540, filed on Dec. 28, 2022.

(51) Int. Cl. F28F 27/00; E04H 4/12; G01N 33/18

(52) U.S. Cl. CPC F28F 27/00; E04H 4/129; G01N 33/1813; F28F 2200/00

(57) ABSTRACT

A device and method of predicting the failure of a heat exchanger of a heating device for a pool or spa are provided. The method comprises providing a measurement device to determine a baseline concentration of a metal within the water of the pool or the spa at a first time. The method further comprises measuring a concentration of the metal via the measurement device at a second time to define a measured concentration and determine if the measured concentration is greater than the baseline concentration. The method also comprises providing an alert when the measured concentration exceeds the baseline concentration by more than a predetermined amount. The metal detected by the measurement device may be at least one of copper, stainless steel, or titanium.

### CROSS-REFERENCE TO RELATED APPLICATIONS

This application claims the benefit of U.S. Provisional Patent Application No. 63/477,540, filed Dec. 28, 2022, entitled SYSTEM AND METHODS FOR DETECTING FAILURE OF HEAT EXCHANGERS, the entire contents of which are incorporated herein by reference.

### FIELD OF INVENTION

The present disclosure relates generally to a system and method for detecting the failure of pool components and more specifically to a system and method for detecting the failure of heat exchangers associated with pool and spa heaters.

### BACKGROUND

Pools and spas typically provide consumers with a source of comfort and entertainment during warm temperature months. To increase the comfort of such consumers during cooler days, cooler months, or in colder geographies, many pools and spas are equipped with heaters. The heaters are provided with heat exchangers adapted to provide thermal energy to the water of the pool and spa, increasing the overall temperature of the pool and spa.

Unfortunately, like all mechanical devices, the heaters and/or components of the heaters (including the heat exchanger) may eventually fail. This can be a source of frustration for pool and spa owners, who may rely on the heater to warm the water of the pool and spa during colder days. To begin, many pool heaters lack protective devices that could be used to prolong the lifetime of the heater. In addition, many heaters do not provide pool and spa owners with adequate warning of an impending failure. The heaters typically lack measurement devices, control systems, or other components that can monitor the performance of the heater such that the pool and spa owners can be provided notice of an impending failure. This also leads to increased frustration from pool and spa owners when the heater malfunctions or stops operating without any warning or explanation.

The present system and methods overcome many of the shortcomings and limitations of the prior art devices discussed above. The systems include several instances of a pool heater provided with a heat exchanger, the heat exchanger designed to warm the water of a pool or spa. The heat exchanger may be composed of at least one metal including, but not limited to, a copper metal, a copper alloy, a stainless steel alloy, a titanium metal, or combinations thereof. Over time, the pool water may corrode the metal of the heat exchanger. In turn, as the heat exchanger corrodes, ions of the corroded metal enter the pool water. Thus, the metal comprising the heat exchanger may be detected in the pool or spa water by a measurement device. In turn, the detection of the metal comprising the heat exchanger in the pool or spa water may serve as an “early warning system” of an impending failure of the heat exchanger.

### SUMMARY

In some instances, the swimming pool heater may be in fluid communication with a sacrificial anode. Preferably, the sacrificial anode is positioned and located upstream of the heat exchanger such that the sacrificial anode may help protect the heat exchanger from corrosion. The sacrificial anode may be designed to decay over time in the pool and spa water to (1) help protect the heat exchanger of the heater from corrosion, and (2) increase a concentration of a compound comprising the sacrificial anode in the pool water. An increase in the concentration of the compound in the pool water can be detected by a measurement device.

The measurement device may be designed to measure a concentration of at least one metal or compound within the water of the pool or spa. In some instances, the measurement device may comprise a colorimeter. The colorimeter may measure the concentration of the metal comprising the heat exchanger and/or the concentration of the compound associated with the decay of the sacrificial anode in the pool and spa water. For example, the colorimeter may be designed to measure the concentration of magnesium, zinc, aluminum, steel, lead, tin, nickel, brass, bronze, copper, and/or other metals in the water of the pool and spa.

The pool heater may also include a controller that is designed to provide an alert to a user when a measured value is detected by the measurement device, or the measured value reaches or exceeds a predetermined threshold. The alert may provide a warning to the user about an impending failure of a component of the heater (e.g., the heat exchanger) and/or a warning that the sacrificial anode is depleted.

In some instances, a sensor may be in electrical or magnetic communication with the sacrificial anode to measure one or more physical parameters associated with the sacrificial anode. For example, the sensor may measure a current flowing through the sacrificial anode, a conductivity value of the sacrificial anode, and/or a mass of the sacrificial anode. An alert may be provided to the user by the sensor (or a controller in communication with the sensor) when one or more physical parameters associated with the sacrificial anode indicate that the sacrificial anode is in a depleted state.

In one aspect, a method of predicting the failure of a heat exchanger of a heating device for a pool or spa is provided. The method includes providing a measurement device such as a colorimeter and determining a baseline concentration of a metal component in the water of the pool or spa via using the colorimeter at a first time period. At a second time period, the colorimeter may again determine the concentration of the metal component to define a measured concentration. The method further includes determining if the measured concentration is greater than the baseline concentration, and providing an alert when the measured concentration exceeds the baseline concentration by more than a predetermined amount.

In some instances, the metal comprises copper. In other instances, the metal comprises at least one of copper, stainless steel, or titanium.

In some instances, the predetermined amount is at least ten percent greater than an initial measurement of the baseline concentration.

In some instances, the method includes a step of providing a controller designed to generate the alert to a user. The alert informs the user of an impending failure of the heat exchanger. In addition, the controller is configured to instruct the measurement device to determine the baseline concentration and to measure the concentration of the metal.

In other instances, the measurement device includes a feed line, a loop having a chemical reagent system, an automated colorimeter, and an outlet line.

In another aspect, a heater for a pool or spa is provided. The heater includes a housing, a heat exchanger, and a sacrificial anode in fluid communication with the heat exchanger. As water is provided to the heater, the sacrificial anode leaches a compound into the water at a predetermined rate. The heater also includes a controller designed to provide an alert to a user when the concentration of the compound in the water reaches a predetermined threshold.

In some instances, the alert informs a user of an impending fault or failure associated with the heat exchanger.

In other instances, the sacrificial anode is selected from the group consisting of a zinc-containing compound, a magnesium-containing compound, an aluminum-containing compound, or combinations thereof. In some instances, the sacrificial anode comprises at least one of magnesium, zinc, aluminum, steel, lead, tin, nickel, brass, or bronze.

In other instances, the sacrificial anode is provided in the form of a ribbon.

In some instances, the sacrificial anode is positioned upstream of the heat exchanger to protect the heat exchanger from corrosion.

In other instances, the heater also includes a measurement device to detect the concentration of the compound in the water.

In some instances, the heater also includes a conductivity meter in electrical communication with the sacrificial anode. The conductivity meter is configured to measure a conductivity value of the sacrificial anode, and the controller provides the alert to the user when the conductivity value of the sacrificial anode reaches a predetermined threshold value.

In yet another aspect, a pool heater is provided. The pool heater comprises a housing, a heat exchanger including a sacrificial anode, and a sensor provided in the form of a conductivity meter in electrical communication with the sacrificial anode. The heater further includes a controller designed to provide an alert to a user when the conductivity value of the sacrificial anode reaches a predetermined threshold.

In a further aspect, a heater for a pool or spa is provided. The heater may be provided in the form of a housing that includes an outlet and an inlet. The heater is in fluid communication via the inlet and outlet. A heat exchanger is retained within the housing and is in fluid communication with the pool or spa via the outlet and the inlet. The heat exchanger is configured to increase a temperature of water of the pool or spa from a first temperature to a second temperature. The heater also includes a measurement device that is positioned downstream of the heat exchanger and is in fluid communication with the outlet. The measurement device is configured to determine a concentration of at least one metal in the water of the pool or spa.

In some instances, the heater also includes a controller designed to receive a signal from the measurement device. In turn, the controller provides an alert when the signal indicates the concentration of the at least one metal in the water of the pool or spa is above a predefined value.

In other instances, the heater includes a controller in electrical communication with the measurement device. The controller provides an alert when at least one of the following conditions occurs: (1) the concentration of the at least one metal in the water of the pool or spa is above a threshold value, (2) the concentration of the at least one metal in the water of the pool or spa is above a baseline concentration of the at least one metal by more than a predetermined amount, or (3) the concentration of the at least one metal in the water of the pool or spa increases at a rate greater than a predetermined rate.

In some instances, a metal of the at least one metal detected by the measurement device is selected from the group consisting of copper, stainless steel, titanium, and combinations thereof.

In other instances, the heater includes a sacrificial anode positioned upstream of the heat exchanger and in fluid communication with the inlet. The sacrificial anode is composed of at least one of magnesium, zinc, aluminum, steel, lead, tin, nickel, brass, and or bronze.

These and other aspects and advantages of the present disclosure will become apparent to those skilled in the art after considering the following detailed description in connection with the accompanying drawings.

### BRIEF DESCRIPTION OF DRAWINGS

FIG. 1 is an isometric view of a heater for a pool or spa;

FIG. 2 is a schematic cross-sectional view of various internal components of the heater of FIG. 1;

FIG. 3 is a block diagram illustrating components of a pool and spa system, the pool and spa system including a measurement device and the heater of FIG. 1;

FIG. 4 is a schematic representation of various components of a controller for the heater of FIG. 1;

FIG. 5 is a schematic diagram representing components of a colorimeter system designed for use with the heater of FIG. 1;

FIG. 6 is a right-side elevational view of various components of an automated colorimeter provided in the colorimeter system of FIG. 5;

FIG. 7 is a schematic cross-sectional view of various internal components of another embodiment of a heater, the heater provided with a sacrificial anode;

FIG. 8 is a top plan view of another embodiment of a sacrificial anode;

FIG. 9 is a schematic representation of a method for detecting an impending failure of a heat exchanger of any of the heaters disclosed herein; and

FIG. 10 is a schematic representation of another method for detecting an impending failure of a heat exchanger of any of the heaters disclosed herein.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION

Before any embodiments are described in detail, it is to be understood that the disclosure is not limited in its application to the details of construction and the arrangement of components set forth in the following description or illustrated in the following drawings, which is limited only by the claims that follow the present disclosure. The disclosure is capable of other embodiments, and of being practiced, or of being carried out, in various ways. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following description is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown but are to be accorded the widest scope consistent with the principles and features disclosed herein. The following detailed description is to be read with reference to the figures, in which like elements in different figures have like reference numerals. Skilled artisans will recognize the examples provided herein have many useful alternatives and fall within the scope of embodiments of the disclosure.

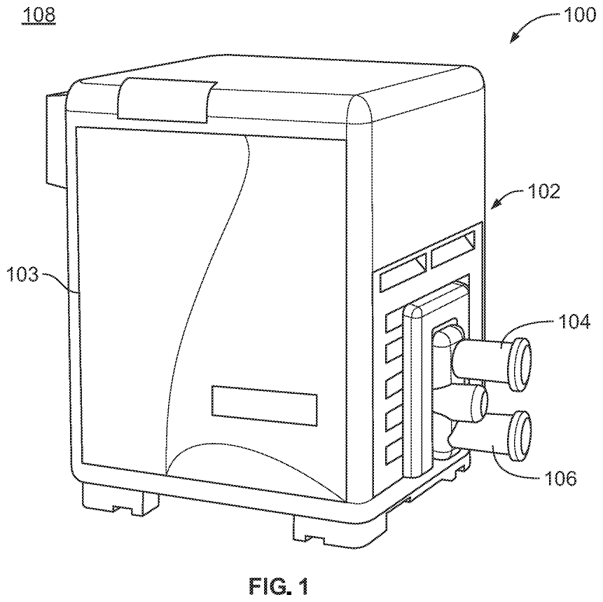

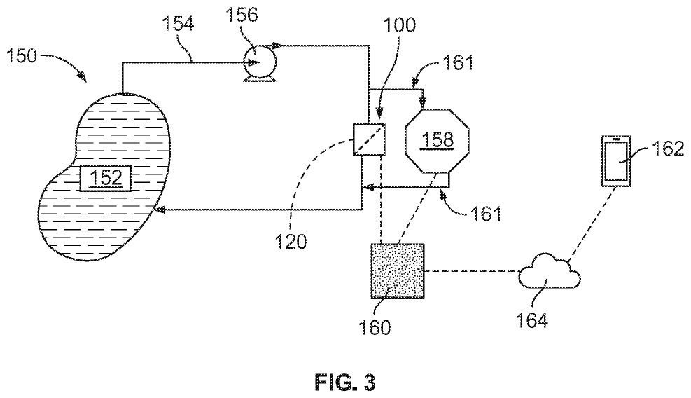

Referring to FIG. 1, a heater 100 is provided. The heater 100 may be provided in the form of a housing 102 defining a body 103, wherein the housing 102 is coupled to an inlet 104 and an outlet 106. Generally, the inlet 104 and the outlet 106 may be in fluid communication with a pool system, a spa system, a combination pool-spa system, such as a pool and spa system 150 illustrated in FIG. 3, or other aquatic applications. The heater 100 may be provided as a heater adapted for use with a pool or a spa, including, by way of example, an electric heater, a gas heater, a condensing heater, and/or a hybrid gas-electric heater. In an embodiment, the heater 100 may be the UltraTemp ETi® Hybrid Heater provided by Pentair, Inc. and described in U.S. Pat. Nos. 9,732,536, 10,400,466, 11,142,923, and 11,686,118, the contents of which are incorporated by reference in their entirety. In another embodiment, the heater 100 may be provided as the MasterTemp® High Performance Pool and Spa Heater, provided by Pentair, Inc. and described in U.S. patent application Ser. No. 17/650,611, the contents of which are incorporated by reference in its entirety.

Referring still to FIG. 1, the body 103 of the housing 102 may be provided substantially in the form of a rectangular prism that substantially covers the components retained within the heater 100. The body 103 may also be provided in other shapes and forms, (e.g., a three-dimensional profile including rounded edges) as would be appreciated by those skilled in the art. In some instances, the body 103 may be composed of a durable material (e.g., metal or plastic) adapted to withstand environmental conditions in an outside environment 108. The components of the heater 100 retained within the body 103 may include a heat exchanger or other heating element, a controller, a blower motor, and the like. The inlet 104 and the outlet 106 may be received into and through the body 103 such that the housing 102 and the components of the heater 100 can be placed in fluid communication with the water of the pool or spa.

The inlet 104 and the outlet 106 may be provided in the form of tubing or pipes that extend outwardly from an interior (not illustrated) of the body 103 and away from the housing 102. The inlet 104 may be in fluid communication with a heating mechanism (not illustrated) within the housing 102, the heating mechanism being one of a heat exchanger, a heat pump, and/or a heating element of the heater 100. The inlet 104 may be adapted to receive water from the pool or spa that is at a first temperature and may provide the water to the heating mechanism to be heated. After the water is provided to the heating mechanism, the temperature of the water may be increased to a second temperature that is greater than the first temperature. After heating, the water may then be provided to the outlet 106 and, eventually, to the pool or spa.

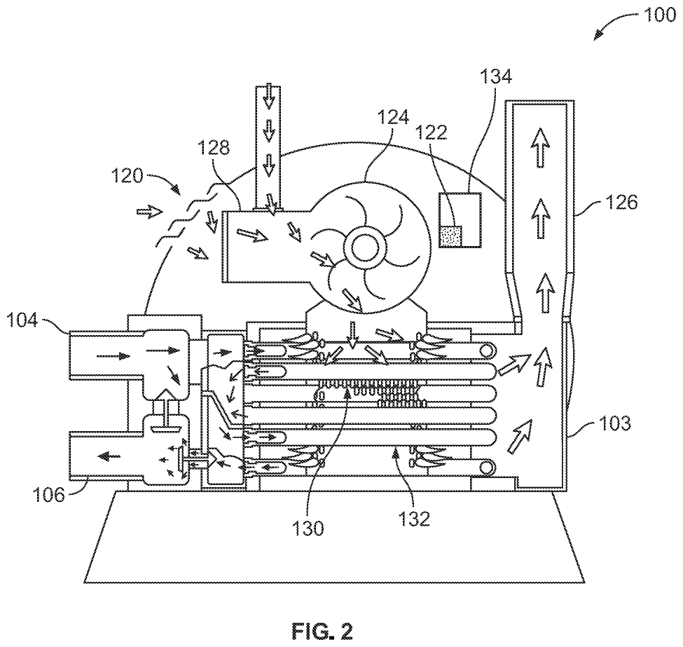

Turning to FIG. 2, in some instances, the heater 100 can include a gas heat exchanger 120, an ignition control board 122, a blower motor 124, an exhaust 126, an air/fuel mixing chamber 128, a burner 130, heating coils 132 and other components associated with the heater 100. The ignition control board 122 is coupled to and controlled by a heater control board 134. For example, the heater control board 134 is configured to activate the blower motor 124 and direct the ignition control board 122 to ignite the burner 130 to engage a heating mode of the heater 100.

In some instances, the blower motor 124 may be provided in the form of a device including vanes or blades that, when activated, cause air or fluids to flow through a conduit. For example, the blower motor 124 may direct air and fuel into air/fuel mixing chamber 128 in preparation for the combustion of the fuel. After the fuel is combusted, the blower motor 124 may also help direct the exhaust gases out of the heater 100 via the exhaust 126.

As the heater 100 operates, water from the pool and spa may be warmed by the heat exchanger 120 within the heating coils 132. The heating coils 132 may be provided in the form of elongated tubes or pipes that are coiled into a helical pattern within the body 103 of the heater 100, although the heating coils 132 may be provided in other shapes and forms. Preferably, the heating coils 132 may comprise copper due to copper's high thermal conductivity, low specific heat, and relatively low cost. In other instances, the heating coils 132 of the heat exchanger 120 may be composed of a copper alloy, a titanium metal, or a stainless steel alloy.

Because the heat exchanger 120, and the heating coils 132 in particular, are exposed to pool and spa water during the operation of the heater 100, the heat exchanger 120 is vulnerable to degradation and corrosion. Over time, the metals comprising the heat exchanger may be oxidized by the water flowing through the heat exchanger 120, which ultimately can lead to failure of the heat exchanger 120. As will be explained in more detail herein, as the heat exchanger 120 corrodes, the concentration of one or more metal or metals from the heat exchanger detected or present in the water of the pool or spa will increase. The concentration change is measured at various time intervals by a measurement device, such as a measurement device 158 (illustrated in FIG. 3), and the measurements can be used to predict an impending failure of the heat exchanger 120.

A controller, such as the heater control board 134 of FIG. 2, a central controller 160 (see FIG. 3), and/or a user device 162 (FIG. 3) can direct the heater 100 to enter one or more modes of operation, including a bypass mode and the heating mode. For example, a valve (not illustrated) can be configured to control a flow of water received from a pool or spa into the inlet 104 based on an operating state identified by the controller. More specifically, the valve may direct the flow of water away from the heater 100 and into a bypass conduit (not illustrated) when the heater 100 is in the bypass mode, and the valve may direct the flow of water into the heater 100 when the heater 100 is in the heating mode. The controller can be configured to transmit control signals that direct the actuation of the valve to achieve the desired operating state. In some instances, the controller can be electrically coupled to at least one sensor or measurement device configured to provide information relating to the pool heater to the controller.

An example method via which the controller can direct the heater 100 to heat the water of the pool or spa is provided. When the heater 100 is in the heating mode, the heater control board 134 may direct (1) the ignition control board 122 to activate the blower motor 124 to begin mixing air and fuel together in the air/fuel mixing chamber 128 and feed the mix of air and fuel to the burner 130, (2) the ignition control board 122 to ignite the burner 130 to combust the mix of air and fuel so that water flowing through the heating coils 132 is heated to the desired temperature, and/or (3) control the amount of water flowing through the heater 100 (and thereby the heating coils 132) by directing the actuation of one or more valves in fluid communication with the heater 100.

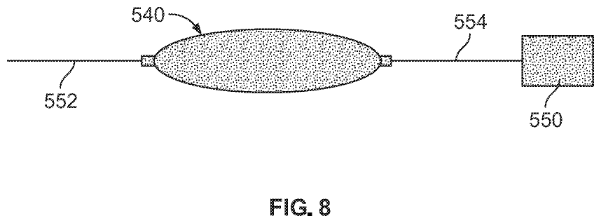

Another example method via which the controller can direct the heater 100 to heat the water of the pool or spa is provided. The heater control board 134 may use inputs from at least one sensor or measurement device (e.g., a measurement device 158 as provided in FIG. 3 and/or a conductivity sensor 550 as provided in FIG. 8), internally stored settings, and/or control signals received from other devices (e.g., local on-board controllers, the central controller 160, and/or the user device 162) to commence various operations. Based on the inputs, the heater control board 134 may be designed to direct (1) the ignition control board 122 to activate the blower motor 124 so as to begin mixing air and fuel together in the air/fuel mixing chamber 128 and feed the mix of air and fuel to the burner 130, (2) the ignition control board 122 to ignite the burner 130 to combust the mix of air and fuel so that water flowing through the heating coils 132 is heated to the desired temperature, and (3) control operation of the valve to one of a plurality of actuation states wherein approximately 100% of the water flowing into the valve flows into the heater 100 and through the heating coils 132.

Although the heater 100 illustrated in FIG. 2 is provided as a heater with a gas heat exchanger, one skilled in the art would understand that the heater 100 may also be provided with an electric heat exchanger or a combination gas-electric heat exchanger.

Turning next to FIG. 3, the pool and spa system 150 provided in the form of a pool or spa 152, fluid conduits 154, a pump 156, the measurement device 158, the heater 100, the central controller 160, and the user device 162. Many of the components of the system 150 are in fluid communication with each other via the conduits 154, which may be provided in the form of tubing or piping adapted to couple the components of the system 150 to each other. Water from the pool or spa 152 may flow through the conduits 154 in the direction indicated by the arrows to form a fluid circuit.

The pump 156 may be provided in the form of a single-speed pump, a two-speed pump, or a variable-speed pump. The pump 156 may be designed to facilitate the flow of water through the system 150 via the conduits 154. For example, the pump 156 may draw water from the pool or spa 152 and then provide the water to the heater 100 and/or the measurement device 158 via the conduits 154. Eventually, the pump 156 may cause the water to return to the pool or spa 152.

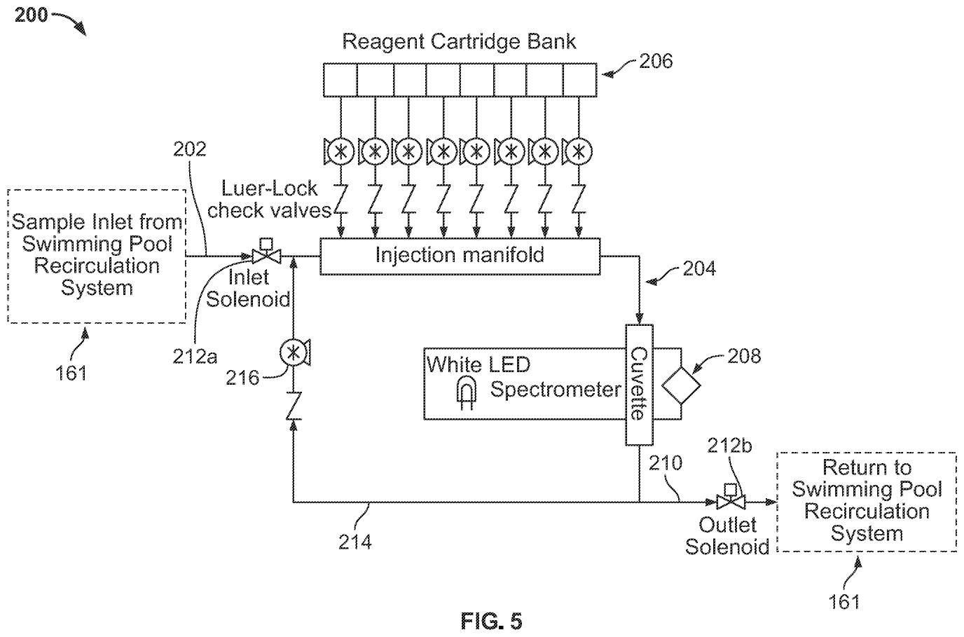

As discussed in greater detail below, the measurement device 158, in conjunction with the central controller 160, may determine when a failure of the heat exchanger 120 is impending by detecting a concentration of a metal within the water of the system 150. In particular, the measurement device 158 may detect the concentration of one or more of magnesium, zinc, aluminum, steel, lead, tin, nickel, brass, bronze, or any combination thereof within the water of the system 150. In some instances, the measurement device 158 may be provided in the form of an ion selective electrode configured to detect the presence of a metal or a combination of metals within the water. In other instances, the measurement device 158 may be provided as a UV-Vis spectrometer configured to detect the concentration of one or more metals within the water. In yet other instances, the measurement device 158 may be provided in the form of a colorimeter, such as a colorimeter system 200, discussed hereinbelow with respect to FIG. 5.

Referring still to FIG. 3, the measurement device 158 may be positioned and located in a bypass line 161. By positioning the measurement device 158 in the bypass line 161, continuous delivery of a fresh sample of water may be provided to the measurement device 158 without the need for additional delivery pumps. In turn, this may reduce the equipment needed to operate the system 150 and thereby reduce equipment maintenance and costs associated with the system 150. Further, by positioning the measurement device 158 in the bypass line 161, the measurement device 158 may sample the water before it passes through the heater 100, which may help ensure that any concentration measurements carried out by the measurement device 158 are properly calibrated. In alternative instances, the measurement device 158 may be coupled to or provided within any of the conduits 154, the heater 100, or the pool or spa 152, as would be appreciated by one skilled in the art.

The user device 162 is configured to interface with the central controller 160, either directly over a local area network or via a cloud network 164. Although FIG. 3 only depicts the central controller 160, the user device 162, and the cloud network 164 in communication with each other, it should be noted that various communication methodologies and connections may be implemented to work in conjunction with, or independent from, one or more local controllers associated with each of the individual components associated with the system 150 (e.g., a controller of the pump 156, a controller 134 of the heater 100, a controller of the colorimeter system 200, etc.).

The central controller 160 may be designed to provide an alert to a user when the measurement device 158 determines that the concentration of a compound in the water of the system 150 reaches or exceeds a predetermined threshold. For example, the central controller 160 may provide the alert to the user device 162 when the concentration of a metal in the water reaches or exceeds a predetermined threshold. The alert may be provided in a manner that informs the user of an impending fault or an impending failure in the heat exchanger 120 of the heater 100. In some forms, the alert may be provided as text or other visual notification, audio, alarm, haptic vibration, or other mechanism that conveys the heater 100 and/or heat exchanger 120 status to the user.

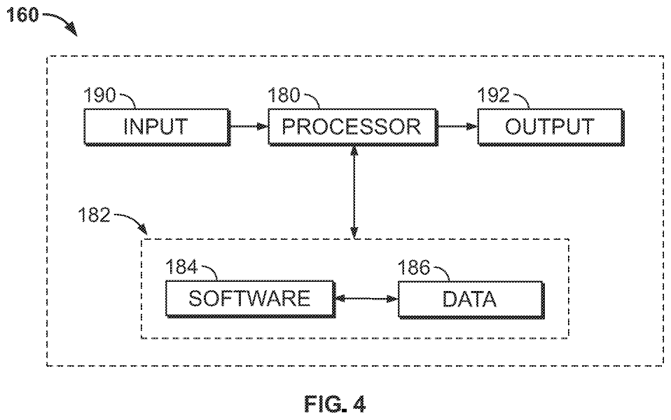

Turning to FIG. 4, the components and modules of the central controller 160 are further illustrated. The central controller 160 may be provided in the form of a processor 180 and a memory 182. The memory 182 includes software 184 and data 186 and is designed for storage and retrieval of processed information to be processed by the processor 180. The processor 180 includes an input 190 that is configured to receive process signals (e.g., signals from the measurement device 158) via the input 190. The central controller 160 may operate autonomously or semi-autonomously, may read executable software instructions from the memory 182 or a computer-readable medium (e.g., a hard drive, a CD-ROM, flash memory), and/or may receive instructions via the input 190 from the user, or another source logically connected to a computer or device, such as another networked computer or server. For example, the server may be used to control the heater 100 or the measurement device 158 via the central controller 160 on-site or remotely.

The processor 180 may process the process signals provided as the input 190 to generate an output 192. The output 192 may take the form of a process control action. Example process control actions may include sending a signal to the heater 100 to route water to the heat exchanger 120, enter the bypass mode, or enter the heating mode; the measurement device 158 to direct the measurement device 158 to measure the concentration of a compound within the water of the system 150; and/or provide an alert to the user device 162 warning of an impending failure in the heat exchanger 120.

As would be understood by one skilled in the art, the components and modules that comprise the central controller 160 could also comprise the heater control board 134, any other controller described herein, or other controllers that may be associated with the system 150.

The central controller 160 may receive a signal or signals from the measurement device 158 related to the concentration of one or more compounds within the water of the system 150. In some instances, a first baseline concentration of a first metal within the water of the system 150 may be measured by the measurement device 158 at a first time period. For example, the metal may comprise copper, stainless steel, titanium, or combinations thereof. The measurement may be received by the central controller 160 as input 190 and/or stored within the memory 182 of the central controller 160. It should be appreciated that the system 150 may repeat this step numerous times to measure, receive, and/or store baseline concentrations of various components (e.g., metals). In other instances, the baseline concentration of one or more components may be input into the system 100 manually or otherwise predefined. The baseline concentrations of the one or more components may be measured and/or defined during a configuration operation of the system 150.

At a second time period, a second measurement of the concentration of the metal may be carried out by the measurement device 158 to define a second measured concentration. In some instances, the second measured concentration may be provided to the central controller 160 as the second input 190 and/or stored within the memory 182 of the central controller 160. Once the second measured concentration is established, the central controller 160 may determine whether the second measured concentration is greater than the baseline concentration. Then, if the central controller 160 determines that the second measured concentration is greater than the baseline concentration, and the second measured concentration exceeds the first baseline concentration by more than a first predetermined amount, the central controller 160 may provide an alert to the user. In some instances, the alert may inform the user of an impending failure of the heat exchanger 120. In other instances, the alert may warn the user of the risk of damage to the heat exchanger 120 if the user continues to operate the heater 100 in the heating mode. It should be appreciated that this process may be repeated for multiple components (e.g., a first metal, a second metal, etc).

In some instances, the first predetermined amount may be at least about 0% greater to at least about 25% greater than the first baseline concentration. For example, the first predetermined amount may be defined as when the second measured concentration is at least about 0% greater, or at least about 1% greater, or at least about 5% greater, or at least about 10% greater, or at least about 15% greater, or at least about 20% greater than the first baseline concentration.

The first baseline concentration detected by the measurement device 158 may at least partially depend on the metal concentration of water from a water source provided to the pool or spa. The water source may include municipal water plants, private water plants, wells, or rainwater. In some instances, the metal concentration (e.g., copper) imparted to the water may depend on the geographic location of the water source. In other instances, the metal concentration imparted to the water may at least partially depend on the composition of the pipes transferring the water from the water source. For example, if the water is delivered from the water source via copper pipes, the water's copper concentration may increase during transport. Ultimately, the metal concentration of the water provided from the water source to the system 150 may be less than about 1 ppm, or less than about 0.5 ppm, or less than about 0.1 ppm, or less than about 0.05 ppm, or less than about 0.01 ppm, or less than about 0.001 ppm, or less than about 0.0001 ppm. In turn, the water of the system 150 may be imparted with a first baseline concentration of about 0 ppm to about 2 ppm.

In some instances, the first baseline concentration may be less than the metal concentration in the water source. This may occur when the water of the pool or spa is treated with a chemical agent that can sequester, chelate, or otherwise remove metals from the water. In such instances, the user may, as input 190, provide information to the central controller 160 indicating that the system 150 has been treated with a metal-removing chemical agent. When such an input is provided, the central controller 160 may direct the measurement device 158 to detect an additional baseline measurement. The additional baseline measurement may replace the first baseline measurement as the reference point by which the central controller 160 decides, using the methods described herein, to provide an alert to the user.

In some instances, the central controller 160 may provide an alert to the user when the second measured concentration of the metal increases at a rate greater than a first predetermined rate. The first predetermined rate may be programmed into the memory 182 of the central controller 160 by a manufacturer of the heater 100, a pool service technician, a pool and spa owner, or another appropriate person or entity. In some cases, the first predetermined rate may be an increase in the metal concentration of at least about 1% to about 10% per a unit of time. In such cases, the unit of time may be, by way of example, an hour, a day, two days, a week, or longer. For example, the first predetermined rate may be an increase in the metal concentration of at least about 1%, or at least about 2%, or at least about 3%, or at least about 5%, or at least about 7%, or at least about 10% per day. As an additional example, the first predetermined rate may be an increase in the metal concentration of at least about 3%, or at least about 5%, or at least about 7%, or at least about 10% per week.

In some instances, a first threshold concentration may be used by the central controller 160 to determine when to provide an alert to the user. The first threshold concentration may be stored in the memory 182 of the central controller 160 or provided to the central controller 160 via a remote device. The first threshold concentration may be determined from information other than the water of the system 150. For example, the first threshold concentration may be based on the average metal concentrations of the water source supplied to the pool or spa. In such instances, an alert may be provided to the user when the measurement device 158 detects that the concentration of the metal is higher than the first threshold concentration. For example, the alert may be provided when the concentration of the metal is at least about 5% greater to at least about 50% greater than the first threshold concentration. As an additional example, the alert may be provided when the measured value of the metal concentration is at least about 5% greater, or at least about 10% greater, or at least about 15% greater, or at least about 20% greater, or at least about 30% greater, or at least about 40% greater, or at least about 50% greater than the first threshold concentration.

In some instances, the first baseline concentration or the first threshold concentration may be less than the detection limit of the measurement device 158. In such instances, the first baseline concentration may be set by the central controller 160 as substantially equivalent to the detection limit of the measurement device 158. As non-limiting examples, the detection limit of the measurement device 158 may be less than about 0.0001 ppm, or less than about 0.0005 ppm, or less than about 0.001 ppm, or less than about 0.005 ppm, or less than about 0.01 ppm, or less than about 0.05 ppm, or less than about 0.1 ppm.

When the first predetermined amount is reached or exceeded, the alert provided to the user by the central controller 160 may be transmitted to the user device 162 via the cloud network 164. The alert may be sent via a text message, a multimedia message, an email notification, an automated phone call, and/or a message provided within a pool automation application. For example, the alert may be provided in the form of a text alert indicating the risk of damage to the heater 100 if the heater is operated in the heating mode. As an additional example, the alert may be provided in the form of a graphical icon indicating the potential impending failure of the heat exchanger 120. In some instances, the notification may be sent to an application executed at the user device 162. The application may be designed to indicate to the user that the heater 100 and/or the heat exchanger 120 needs servicing by a technician. The application executed at the user device 162 may be provided in the form of an application developed by a manufacturer or a supplier of the heater 100.

When the central controller 160 determines that there is a risk of damage to the heat exchanger 120 or that the failure of the heat exchanger 120 is impending, the central controller 160 may be designed to place one or more functions of the central controller 160 into a lockout mode. The lockout mode may disable one or more functions of the central controller 160 until the heater 100 is serviced by a technician. In some instances, a first function of the one or more functions is operating the heater 100 in the heating mode. Thus, the central controller 160 may prevent or temporarily disable the ability of the heater 100 to operate in the heating mode when the central controller 160 is in the lockout mode. Such lockout modes may be designed to help protect the various components of the heater 100, including the heat exchanger 120, from degradation. In addition, the alert may be sent to the user device 162 indicating that the heater 100 has been temporarily disabled.

In some instances, the user may override the lockout mode of the central controller 160 to re-enable the one or more functions. In such instances, a notification or alert may be provided to the user device 162 warning the user of the risk of damage to the heater 100 if the lockout mode is disabled.

In some instances, the alert provided by the central controller 160 may be provided to a pool service person.

In some instances, the alert may not be provided to the user and/or the heater 100 may not enter into the lockout mode until a set number of measurements confirm that the metal concentration in the water of the system 150 is increasing relative to a baseline concentration. In such instances, the alert may not be provided to the user until at least two measurements (either consecutive or nonconsecutive) confirm the concentration of the metal in the water is increasing. For example, the alert may not be provided to the user until three consecutive measurements carried out by the measurement device 158 have confirmed that the measured value is above the first baseline concentration by the first predetermined amount. As an additional example, the alert may not be provided to the user until two consecutive measurements carried out by the measurement device 158 have confirmed that the metal concentration is increasing by at least the first predetermined rate.

Referring now to FIG. 5, components of the colorimeter system 200 are illustrated. As previously stated, the colorimeter system 200 may be provided as the measurement device 158 in the system 150 of FIG. 3. The colorimeter system 200 may include a feed line 202, a loop 204 comprising a chemical reagent system 206, an automated colorimeter 208, and an outlet line 210. The feed line 202 may permit a sample of water from the system 150 to flow into the loop 204 and the feed line 202 may be in fluid communication with the bypass line 161 upstream of the heater 100 (see FIG. 3). The feed line 202 may include a first solenoid valve 212a. The first solenoid valve 212a may control the flow of water into the loop 204.

Referring still to FIG. 5, the chemical reagent system 206 may be positioned within the loop 204 downstream of the first solenoid valve 212a. The chemical reagent system 206 may control the dosage rate of one or more chemical reagents injected into the water sample flowing through the loop 204. The automated colorimeter 208 may be positioned downstream of the chemical reagent system 206. After the water sample passes through the chemical reagent system 206 and the automated colorimeter 208, the water may return to the feed line 202 via a return line 214 and/or exit the loop 204 via the outlet line 210. The return line 214 may be connected to the feed line 202 downstream of the first solenoid valve 212a and upstream of the chemical reagent system 206. The return line 214 may comprise a pump 216, which may be a recycle pump and/or a mixing pump that mixes the one or more reagents injected into the loop 204 with the sample of water.

The outlet line 210 may be coupled to the system 150. The outlet line 210, like the feed line 202, may be provided to the bypass line 161 (in this case, the outlet line 210 is downstream of the heater 100). The outlet line 210 may comprise a second solenoid valve 212b and the second solenoid valve 212b may control the flow of the water out of the loop 204.

When the colorimeter system 200 carries out a measurement operation, the first solenoid valve 212a may be opened to permit a water sample to flow into the loop 204. One or more chemical reagents may also be injected into the water sample via the chemical reagent system 206. The one or more chemical reagents may mix with the water sample prior to the water sample entering the automated colorimeter 208. As the mixed water sample passes through the automated colorimeter 208, the mixed water sample is measured to determine a value of one or more water quality parameters. Preferably, the one or more water quality parameters are related to the concentration of a metal within the water sample. The one or more reagents injected into the loop 204 may depend on the one or more water quality parameters being tested. Preferably, the one or more chemical reagents provided in the chemical reagent system 206 comprise compounds that can help the colorimeter system 200 detect the presence of a metal (e.g., copper, stainless steel, and/or titanium) in the water sample. The mixed water sample then exits the colorimeter system 200 and may continue to circulate through the loop 204 via the return line 214 and/or exit the loop 204 via the outlet line 210 when the second solenoid 212b is in an open position.

During a mixing operation, the first and second solenoid valves 212a, 212b may be closed to isolate the water sample to the loop 204. The pump 216 may reverse the flow direction of the water sample to mix the water sample and the one or more reagents injected into the loop 204.

During a rinsing operation, the first and second solenoid valves 212a, 212b may be open, which may allow for a continuous flow of water through the portion of the colorimeter system 200 comprising the chemical reagent system 206 and the automated colorimeter 208. The rinsing mode may remove samples that have been analyzed and deliver fresh sample water for additional analysis. The pump 216 may be turned on and off occasionally while in the rinse mode. This may help ensure that a fresh sample of water is consistently present in each portion of the colorimeter system 200. Moreover, the pump 216 may help remove prior reagents from the colorimeter system 200.

Each of the solenoid valves 212a, 212b described in FIG. 5 may be direct-acting solenoid valves, indirect-acting solenoid valves, normally closed or normally open solenoid valves, and any combination thereof.

The actions of the colorimeter system 200 may be directed by the central controller 160. In such instances, the central controller 160 may provide instructions or signals to the colorimeter system 200 such that the colorimeter system 200 will carry out the steps described above. In addition, in some instances, the measured values obtained by the colorimeter system 200 (e.g., a metal concentration in the water sample) are provided to the central controller 160, wherein the measured values can be used as the input 190 or stored within the memory 182. In some instances, the colorimeter system 200 may be provided with a secondary controller (not illustrated) that may direct the operations of the colorimeter and/or process the measured values obtained by the colorimeter system 200.

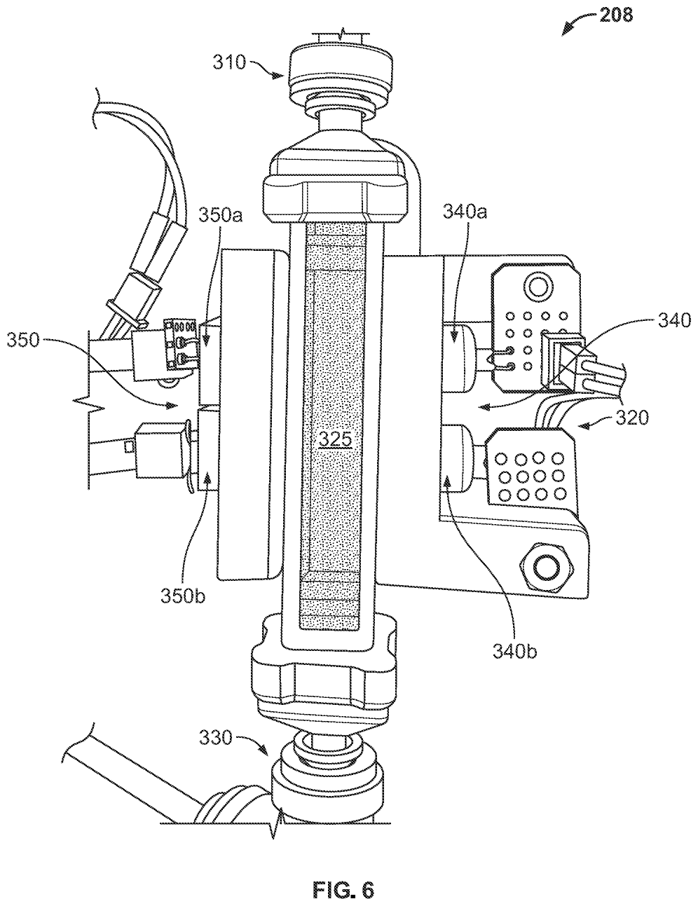

FIG. 6 illustrates a detailed view of an embodiment of the automated colorimeter 208. The automated colorimeter 208 may be provided in the form of a photodetector comprising an inlet 310, a cuvette system 320, and an outlet 330. A pool water sample may flow into the cuvette system 320 through the inlet 310 and flow out of the cuvette system 320 through the outlet 330.

The cuvette system 320 may be provided in the form of a vial 325, one or more light sources 340, and one or more light collectors 350. As shown, the cuvette system 320 may comprise a first light source 340a, a second light source 340b, a first light collector 350a, and a second light collector 350b. In alternative instances, the cuvette system 320 may comprise more or fewer light sources 340 and light collectors 350. The one or more light sources 340 may comprise a colored LED of selected color and wavelength and/or a white LED to analyze the developed reagent chemistry of the sample water after one or more chemical reagents have been injected into a loop of a chemical reagent system, such as the chemical reagent system 206 of FIG. 5.

The vial 325 may be provided in the form of an at least partially transparent material that permits at least some light to pass through the vial 325 with little to no interference such as Pyrex® glass, UV quartz, IR quartz, sapphire, or an optically clear polymer such as polystyrene, acrylic, or polycarbonate. To measure the one or more water quality parameters, the cuvette system 320 may be configured to shine a light from the one or more light sources 340 through the vial 325 holding a water sample, and the one or more light collectors 350 may measure the intensity of the light that passes through the water sample in the vial 325.

Referring still to FIG. 6, during the operation of the automated colorimeter 208, one or more chemical reagents may be combined with the water sample during measurement of the one or more water quality parameters. Further, the automated colorimeter 208 may provide information on reagent degradation by comparing the results of a first and second measurement. For example, when performing a Total Chlorine test, the addition of an iodide reducing agent and subsequent formation of colored iodine solution may indicate the presence of chlorine and may be used to distinguish between a true zero free chlorine reading or a false zero free chlorine reading due to bleaching of the reagent. In another example, an ORP measurement may be used to distinguish between a true zero free chlorine reading or a false zero free chlorine reading due to bleaching of the reagent.

Preferably, the one or more water quality parameters measured by the automated colorimeter 208 is the concentration of at least one metal. Particularly preferably, the at least one metal measured by the automated colorimeter 208 is magnesium, zinc, aluminum, steel, lead, tin, nickel, brass, bronze, or combinations thereof. If the automated colorimeter 208 is determining the concentration and/or presence of at least one metal, the one or more chemical reagents delivered to the water sample by the chemical reagent system 206 may be 1,4-dioxane, 4-(2′-benzothiazolylazo)salicylic acid, 2,4-dinitro APTPT, diethyldithiocarbamate, 2-amino-4-(m-tolylazo)pyridine-3-ol, 2-(5-bromo-2-oxoindolin-3-ylidene) hydrazine carbothioamide, 4-(4′-nitrobenzylideneimino)-3-methyl-5-mercapto-1,2,4-triazole, 2-acetylpyridine thiosemicarbazone, N,Nbissalicylidene-2,3-diaminopyridine, rhodamine derivative with 5-(\acute{α}-methyl-3 hydroxybenzylidene), and/or cupferron. The reagents may help generate a spectroscopic-compatible water sample that can be analyzed by the automated colorimeter 208.

When the measurement of one or more water quality parameters is complete, the water sample may be discharged from the cuvette system 320 via the outlet 330. The outlet 330 may be connected to a loop or bypass such as the bypass line 161 of FIG. 3, the loop 204 of FIG. 5, and/or a waste container.

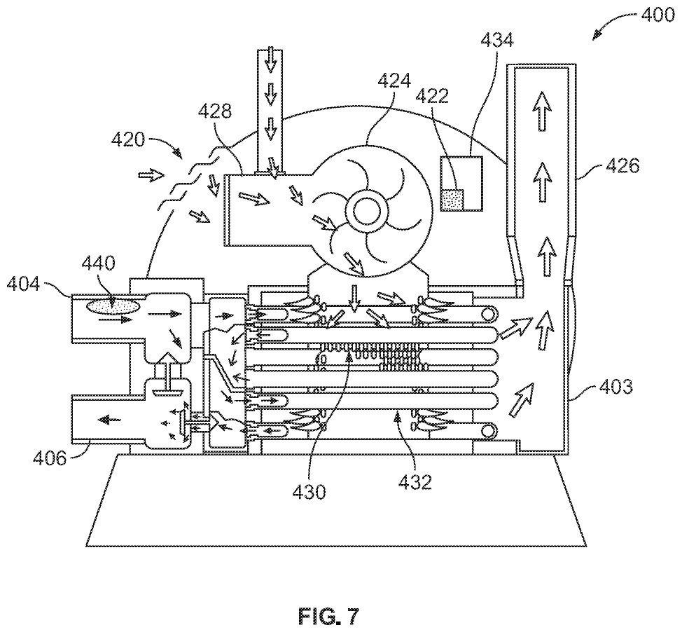

Turning next to FIG. 7, another embodiment of the heater 400 for heating water of a pool or spa is provided. The heater 400 may have a substantially similar function and operation as the heater 100 and may be incorporated in the system 150 (described with reference to FIG. 3). In addition, the embodiment of FIG. 7 may be provided in the form of a body 403, an inlet 404, an outlet 406, a gas heat exchanger 420, an ignition control board 422, a blower motor 424, an exhaust 426, an air/fuel mixing chamber 428, a burner 430, heating coils 432, and a heater control board 434 having substantially the same function and configuration as the similarly named components described with reference to FIGS. 1 and 2. Further, like the heater 100, the heater 400 may be in electrical communication with the central controller 160 (see FIGS. 3 and 4). Unlike the heater 100, the heater 400 may be provided with, coupled to or in fluid communication with a sacrificial anode 440.

The sacrificial anode 440 may be provided in the form of a disc, an elongated cylinder, a sphere, a rectangular prism, a U-shaped member, an elongated wire, a ribbon, or other three-dimensional shapes. The sacrificial anode 440 may be provided in any position in the system 150 of FIG. 3 that is upstream of the heater 400 or the heat exchanger 420. Preferably, the sacrificial anode 440 is coupled to a fluid conduit within the heater 400 that is upstream of the heat exchanger 420, such as the inlet 404, as illustrated in FIG. 7. Further, the sacrificial anode 440 may be coupled to an electrically conductive surface or component of the heater 400 that allows for the free exchange of electrons to and from the sacrificial anode 440. For example, the sacrificial anode 440 may be coupled to a bonding lug (not illustrated) of the heater 400.

The sacrificial anode 440 may comprise any metal that is more active than the metal of the heat exchanger 420. For example, the sacrificial anode 440 may comprise zinc, magnesium, and/or aluminum. As an additional example, the sacrificial anode 440 is selected from the group consisting of a zinc-containing compound, a magnesium-containing compound, an aluminum-containing compound, and combinations thereof. As yet another example, the sacrificial anode 440 may comprise at least one of magnesium, zinc, aluminum, steel, lead, tin, nickel, brass, and bronze. The purpose of the sacrificial anode 440 is two-fold: (1) the sacrificial anode may protect the heat exchanger 420 from degradation or corrosion, which may prolong the life of the heat exchanger 420, and (2) a rate of decay of the sacrificial anode 440 may be monitored to serve as an early-warning system to indicate that the sacrificial anode 440 needs replacement and that the heat exchanger 420 is at risk for damage from corrosion.

As the sacrificial anode 440 is consumed by the water of a pool or spa, the concentration of the metal or compound comprising the sacrificial anode 440 in the water may increase. The increase of the concentration of the metal or compound in the water may be detected by the measurement device 158 (described with reference to FIG. 3). If the concentration of the metal or compound comprising the sacrificial anode 440 increases above a second predetermined value, at a rate greater than a second predetermined rate, or above a second threshold concentration, the central controller 160 may provide a signal to the user device 162 that the sacrificial anode 440 is in a depleted state and needs replacement. Like the first predetermined amount discussed with reference to FIGS. 3 and 4, the predetermined value can be determined by taking a baseline measurement of the metal or compound comprising the sacrificial anode 440 at a first time and comparing the baseline measurement to a concentration measurement carried out at a second time. In some instances, the predetermined value may be a percentage by which the measured concentration exceeds a second baseline concentration. For example, the predetermined value may be defined as when the measured concentration exceeds the second baseline concentration by at least about 2% to at least about 20%. As an additional example, the predetermined value may be when the measured concentration exceeds the second baseline concentration by at least about 5%, or at least about 7%, or at least about 10%, or at least about 12%, or at least about 15%.

Similar to the first predetermined rate discussed with reference to FIGS. 3 and 4, the second predetermined rate can be determined by monitoring the rate by which the metal concentration of the water of the pool and spa increases. In some instances, the second predetermined rate may be an increase in the metal concentration of at least about 1% to about 10% per a unit of time. For example, the predetermined rate may be an increase in the metal concentration of at least about 1%, or at least about 2%, or at least about 3%, or at least about 5%, or at least about 7%, or at least about 10% per day. As an additional example, the predetermined rate may be an increase in the metal concentration of at least about 3%, or at least about 5%, or at least about 7%, or at least about 10% per week.

Like the first threshold concentration discussed with reference to FIGS. 3 and 4, the threshold value may at least partially depend on the concentration of the metal in the water source provided to the pool and spa. The second threshold concentration may either be preprogrammed into the central controller 160 or provided to the central controller 160 by a remote device. An alert informing the user that the sacrificial anode 440 is in a depleted state may be provided when the measurement device 158 detects that the concentration of the metal is higher than the second threshold concentration. For example, the alert may be when the measured value of the metal is at least about 5% greater to at least about 25% greater than the threshold concentration. As an additional example, the alert may be provided when the metal concentration is at least about 5% greater, or at least about 10% greater, or at least about 15% greater, or at least about 20% greater than the threshold concentration.

In some instances, the sacrificial anode 440 is designed to leach a compound into the water of a pool or a spa at a third predetermined rate. The third predetermined rate may be directly proportional to the mass lost by the sacrificial anode 440 as the sacrificial anode 440 is corroded or degraded by the water of the pool and spa. For example, the sacrificial anode 440 may leach no more than about 40%, or no more than about 50%, or no more than about 60%, or no more than about 70%, or no more than about 80%, or no more than about 85%, or no more than 90% of its initial mass into the water of the pool and spa over a time period of one year. As an additional example, the sacrificial anode 440 may leach no more than about 70% to about 90% of its mass over two years. In such instances, the increase in the concentration of the compound comprising the sacrificial anode 440 may increase in the pool or the spa in a predictable manner. Thus, if the central controller 160 is provided with information related to the predetermined rate, the central controller 160 may be configured to provide information related to an estimated remaining lifetime of the sacrificial anode 440 (i.e., the amount of time remaining before the sacrificial anode 440 needs replacement). This information may be provided to the user via the user device 162 in real-time such that the user can determine when he or she should purchase a new sacrificial anode 440.

In some instances, when the predetermined value is reached or exceeded, a signal may be provided to the central controller 160 by the measurement device 158. When the central controller 160 receives a signal from the measurement device 158 indicating that the sacrificial anode 440 is in the depleted state, the central controller 160 may transmit a notification to the user device 162 via the cloud network 164. The notification may be sent via a text message, a multimedia message, an email notification, an automated phone call, and/or a message provided within a pool automation app. For example, the notification may be provided in the form of a text alert. As an additional example, the alert may be provided in the form of a graphical icon that indicates that the sacrificial anode 440 is depleted. In some instances, the notification may be sent to an application executed at the user device 162. The application may be designed to indicate to the user that the sacrificial anode 440 needs replacement. The application executed at the user device 162 may be provided in the form of an application developed by a manufacturer or a supplier of the heater 100. In some instances, the alert provided by the central controller 160 may be provided to a pool serviceperson.

When the central controller 160 determines that the sacrificial anode 440 is in the depleted state, the central controller 160 may be designed to place one or more functions of the central controller 160 into a lockout mode until the sacrificial anode 440 is replaced. In some instances, a first function of the one or more functions is operating the heater 400 in a heating mode. Thus, the heating mode of the heater 400 may be temporarily disabled by the central controller 160 when the central controller 160 is in the lockout mode. The lockout mode may be designed to help protect the heat exchanger 420 from degradation after the sacrificial anode 440 is depleted. In addition, an alert may be sent to the user device 162 indicating that the heater 400 has been temporarily disabled until the sacrificial anode 440 is replaced.

In some instances, the user may override the lockout mode of the central controller 160 to re-enable the one or more functions. In such instances, a notification or alert may be provided to the user device 162 warning the user of the risk of damage to the heater 400 and/or the heat exchanger 420 if the lockout mode is disabled.

In some instances, the central controller 160 may not be in communication with the heater 400. In such instances, the heater control board 134 of the heater 100 may execute any of the functions and processes of the central controller 160 described herein.

Turning now to FIG. 8, another embodiment of a sacrificial anode 540 is illustrated. The sacrificial anode 540 may have a substantially similar function and operation as the sacrificial anode 440. However, unlike the sacrificial anode 440, the sacrificial anode 540 may be provided in the form of an elongated wire, rod, or ribbon that is in electrical communication with the conductivity sensor 550.

The conductivity sensor 550 may be designed to detect the conductivity of the sacrificial anode 540 when an electrical current is applied to the sacrificial anode 540 via wires 552 and 554. When the sacrificial anode 540 is not depleted (i.e., when the sacrificial anode 540 is in a use state) the conductivity of the sacrificial anode 540 may be a non-zero value. In comparison, when the sacrificial anode 540 is in a depleted state, the conductivity of the sacrificial anode 540 may drop to an about zero value, or zero value. When the conductivity of the sacrificial anode 540 drops to the about zero value or zero value, the conductivity sensor 550 may send a signal to the central controller 160 indicating that the sacrificial anode 540 is in the depleted state. The central controller 160 may then provide an alert to the user indicating the sacrificial anode 540 is in the depleted state, in accordance with the teachings provided herein.

Referring still to FIG. 8, in some instances, the conductivity sensor 550 may also be designed to measure the current flowing through the sacrificial anode 540. In such instances, when the sacrificial anode is in the use state, the current flowing through the sacrificial anode 540 may be a non-zero value. Then, when the sacrificial anode 540 is in the depleted state, the value of the current flowing through the sacrificial anode 540 may be about zero, or zero. The conductivity sensor 550 may detect this change in the current flowing through the sacrificial anode 540 and send a signal to the central controller 160 indicating that the sacrificial anode 540 is in the depleted state. The central controller 160 may then provide an alert to the user indicating the sacrificial anode 540 is in the depleted state, in accordance with the teachings provided herein.

In some instances, the conductivity sensor 550 may not be provided. Instead, the decay of the sacrificial anode 540 may be monitored via a mass sensor (not illustrated) that is coupled to the sacrificial anode 540. The mass sensor may monitor a mass of the sacrificial anode over time. Like the conductivity sensor 550, the mass sensor may provide a signal to the central controller 160 when the sacrificial anode 540 is in the depleted state. To determine when the sacrificial anode 540 is in the depleted state, the mass sensor may determine a deployment mass of the sacrificial anode 540 at a first time when the sacrificial anode 540 is first deployed. Then, at a later time (e.g., second time), the mass sensor may measure the current mass of the sacrificial anode 540. After obtaining the current mass measurement, the mass sensor may provide a signal to the central controller 160 indicating that the sacrificial anode 540 is in the depleted state when the current mass is less than about 25% of the deployment mass. For example, the mass sensor may provide a signal to the central controller 160 when the current mass of the sacrificial anode 540 is less than about 20%, or less than about 15%, or less than about 10%, or less than about 5%, or about 0% of the deployment mass of the sacrificial anode 540. As an additional example, the mass sensor may provide a signal to the central controller 160 when the current mass of the sacrificial anode is about zero grams.

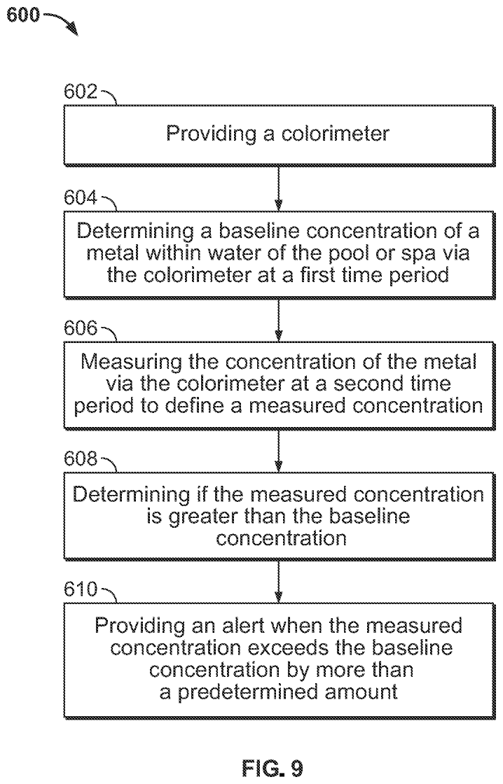

As illustrated in FIG. 9, a method 600 of predicting the failure of a heat exchanger of a heating device for a pool or spa is provided. The method 600 may comprise a step 602 of providing a colorimeter (or other sensor or chemical measurement device). The method 600 may also comprise a step 604 of determining a baseline concentration of a metal within the water of the pool or spa via the colorimeter at a first time. The method 600 may further comprise a step 606 of measuring the concentration of the metal via the colorimeter at a second time to define a measured concentration. The method 600 may also comprise a step 608 of determining if the measured concentration is greater than the baseline concentration. The method 600 may also comprise a step 610 of providing an alert when the measured concentration exceeds the baseline concentration by more than a predetermined amount.

In some instances of the method 600, the metal detected by the colorimeter comprises at least one of copper, stainless steel, and titanium. In other instances, the metal comprises copper. In yet other instances, the metal detected by the colorimeter comprises at least one of magnesium, zinc, aluminum, steel, lead, tin, nickel, brass, or bronze. In some instances, the predetermined amount is at least ten percent greater than an initial measurement of the baseline concentration.

It should be appreciated that one or more steps may be omitted and/or repeated. For example, if the measured concentration of the metal is equal to, substantially equal to, or does not exceed the baseline concentration by more than the predetermined amount, the method will return to step 606 and measure the concentration of the metal at a later time period. The method may continue in this loop until step 610 is triggered. The time period that the system may measure the concentration at step 606 may be once an hour, once a day, once a week, once a month, or another time period.

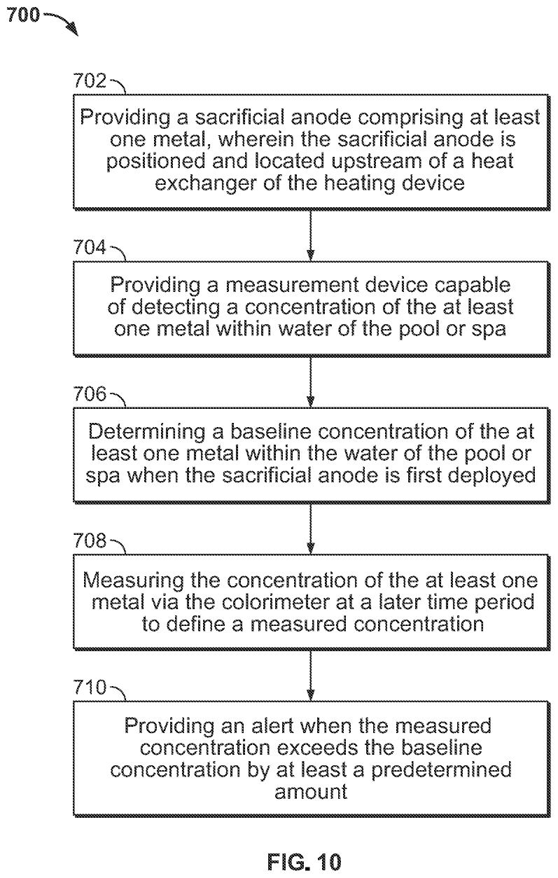

As illustrated in FIG. 10, a method 700 of predicting the failure of a heat exchanger of a heating device for a pool or spa is provided. The method 700 may comprise a step 702 of providing a sacrificial anode comprising at least one metal, wherein the sacrificial anode is positioned and located upstream of a heat exchanger of the heating device. The method 700 may further comprise a step 704 of providing a measurement device (e.g., colorimeter, sensor, and the like) capable of detecting a concentration of the at least one metal within the water of the pool or spa. The method 700 may further comprise a step 706 of determining a baseline concentration of the at least one metal within the water of the pool or spa when the sacrificial anode is first deployed. The method 700 may further comprise a step 708 of measuring the concentration of the at least one metal via the measurement device at a later time to define a measured concentration. The method 700 may further comprise a step 710 of providing an alert when the measured concentration exceeds the baseline concentration by at least a predetermined amount.

In some instances of the method 700, the measurement device is provided in the form of a colorimeter. In other instances of the method 700, the at least one metal of the sacrificial anode is selected from the group consisting of a zinc-containing compound, a magnesium-containing compound, an aluminum-containing compound, or combinations thereof. In yet other instances of the method 700, the sacrificial anode comprises at least one of magnesium, zinc, aluminum, steel, lead, tin, nickel, brass, or bronze. In some instances of the method 700, the sacrificial anode is provided in the form of a ribbon.

The methods 600, 700 may further comprise additional steps consistent with the teachings disclosed herein. In addition, the methods 600, 700 may comprise fewer steps than those described herein.

Additionally, while the following discussion may describe features associated with specific devices or instances, it is understood that additional devices and/or features can be used with the described systems and methods and that the discussed devices and features are used to provide examples of possible instances, without being limited.

It is to be understood that the controllers, other connected devices, and sensors (e.g., the control boards 122, 134, 422, 434, the measurement device 158, the central controller 160, the conductivity sensor 550 and/or the user device 162) as disclosed herein can include respective transceiver and memory devices, each of which can be in communication with control circuitry, one or more programmable processors, and executable control software as would be understood by one of ordinary skill in the art. In some instances, the control software can be stored on a transitory or non-transitory computer readable medium, including, but not limited to local computer memory, RAM, optical storage media, magnetic storage media, flash memory, and the like, and some or all of the control circuitry, the programmable processors, and the control software can execute and control at least some of the methods described herein.

It will be appreciated by those skilled in the art that while the above disclosure has been described above in connection with particular embodiments and examples, the above disclosure is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the above disclosure are set forth in the following claims.

1. A method of predicting failure of a heat exchanger of a heating device for a pool or spa, comprising:
providing a measurement device;
determining a baseline concentration of a metal within water of the pool or the spa via the measurement device at a first time to define a baseline concentration;
measuring a concentration of the metal via the measurement device at a second time to define a measured concentration;
determining if the measured concentration is greater than the baseline concentration; and
providing an alert when the measured concentration exceeds the baseline concentration by more than a predetermined amount.

2. The method of claim 1, wherein the metal comprises copper.

3. The method of claim 1, wherein the heat exchanger includes a metallic component, and the metallic component comprises at least one of copper, stainless steel, or titanium.

4. The method of claim 1, wherein the predetermined amount is at least ten percent greater than the baseline concentration.

5. The method of claim 1, wherein the measurement device is provided in the form of a colorimeter.

6. The method of claim 1 further comprising providing a controller configured to instruct the measurement device to determine the baseline concentration and to measure the concentration of the metal.

7. The method of claim 1, wherein the measurement device is provided in the form of at least one of a feed line, a loop having a chemical reagent system, an automated colorimeter, and an outlet line.

8. A heater for a pool or a spa, comprising:
a housing;
a heat exchanger coupled to a sacrificial anode, wherein the sacrificial anode leaches a compound into water at a predetermined rate; and
a controller designed to provide an alert to a user when a concentration of the compound in the water reaches a predetermined threshold.

9. The heater of claim 8, wherein the alert informs the user of an impending fault in the heat exchanger.

10. The heater of claim 8, wherein the sacrificial anode is selected from the group consisting of a zinc-containing compound, a magnesium-containing compound, an aluminum-containing compound, or combinations thereof.

11. The heater of claim 8, wherein the sacrificial anode comprises at least one of magnesium, zinc, aluminum, steel, lead, tin, nickel, brass, or bronze.

12. The heater of claim 8, wherein the sacrificial anode is provided in the form of a ribbon.

13. The heater of claim 8, wherein the sacrificial anode is positioned upstream of the heat exchanger to protect the heat exchanger from corrosion.

14. The heater of claim 8, wherein a measurement device is provided to detect the concentration of the compound in the water and is in electrical communication with the heater.

15. The heater of claim 8 further comprising a conductivity meter in electrical communication with the sacrificial anode, the conductivity meter configured to measure a conductivity value of the sacrificial anode, and wherein the controller provides the alert to the user when the conductivity value of the sacrificial anode reaches a predetermined threshold value.

16. A heater for a pool or spa, comprising:
a housing including an inlet and an outlet, the inlet and the outlet each in fluid communication with the pool or spa;
a heat exchanger retained within the housing, the heat exchanger in fluid communication with the inlet and the outlet and configured to increase a temperature of water of the pool or spa; and
a measurement device positioned downstream of the heat exchanger and in fluid communication with the outlet, the measurement device configured to determine a concentration of at least one metal associated with the heat exchanger in the water of the pool or spa.

17. The heater of claim 16 further including a controller designed to receive a signal from the measurement device, wherein the controller provides an alert when the signal indicates the concentration of the at least one metal in the water of the pool or spa is above a predefined value.

18. The heater of claim 16 further including a controller in electrical communication with the measurement device, wherein the controller provides an alert when at least one of the following conditions occurs:
(1) the concentration of the at least one metal in the water of the pool or spa is above a threshold value;
(2) the concentration of the at least one metal in the water of the pool or spa is above a baseline concentration of the at least one metal by more than a predetermined amount; or
(3) the concentration of the at least one metal in the water of the pool or spa increases at a rate greater than a predetermined rate.

19. The heater of claim 16, wherein a metal of the at least one metal detected by the measurement device is selected from the group consisting of copper, stainless steel, titanium, and combinations thereof.

20. The heater of claim 16 further including a sacrificial anode positioned upstream of the heat exchanger and in fluid communication with the inlet, and wherein the sacrificial anode comprises at least one of magnesium, zinc, aluminum, steel, lead, tin, nickel, brass, or bronze.
